(19) United States
(12) Patent Application Publication
(10) Pub. No.: US 2005/0149030 A1
(43) Pub. Date: Jul. 7, 2005

(54) Facet joint fixation system

(75) Inventors: Hassan Serhan, South Easton, MA (US); Alexandre Michel DiNello, Cotuit, MA (US); Michael Andrew Slivka, Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: DePuy Spine, Inc., Raynham, MA (US)

(21) Appl. No.: 10/742,193

(22) Filed: Dec. 19, 2003

(51) Int. Cl. A61B 17/56

(52) U.S. Cl. 606/73

(57) ABSTRACT

A facet joint fixation system that provides initial rigidity for stabilizing the facet joint, is sufficiently strong to withstand facet joint shear stresses, and (after supporting a successful fusion) can be resorbed by the body.

### BACKGROUND OF THE INVENTION

Current spine fusion procedures rely heavily on the use of anteriorly-placed cages and posterior fixation to achieve the necessary stability and rigidity to obtain successful clinical results. Accordingly, conventional posterior instrumentation used to augment a spinal fusion primarily includes metallic facet screws, translaminar facet screws, and postero-lateral pedicle screw-plate systems. In some system, rods replace the above-mentioned plates. Because implantation of posterior instrumentation including pedicle screws necessarily involves removing important musculoskeletal elements, there is heightened interest in developing improved systems based upon facet screws.

Facet screws have long been used as an alternative to pedicle fixation screws. For example, Published PCT Patent Application No. WO 00/62684 (“Marino”) discloses a method of securing facet joints by drilling a hole through the joint and inserting a conventional facet screw. U.S. Pat. No. 5,527,312 (“Ray”) discloses a system comprising a conventional facet screw coupled with a securement hook that wraps around the superior transverse process.

In each case, the facet screw has the shape of a conventional plain screw. Accordingly, the threaded portions thereof may be vulnerable under high shear stresses such as those present near the facet joint space.

Deguchi et al., Spine, 23(12):1307-1313 (1998) teaches bioabsorbable poly-L-lactic acid interference pins adapted for trans-laminar facet fixation. Deguchi reported using smooth surfaced pin in its experiments, and the results reported therein showed that although these smooth pins provided more rigidity than the intact FSUs (of sheep), they provided significantly less rigidity than translaminar cortical screws or pedicle screw constructs. Since Deguchi also appears to contemplate PLLA screws, Deguchi appears to disclose a resorbable pin having either a completely smooth shaft or a completely threaded (screw-like) shaft. Accordingly, these devices have the weaknesses discussed above. In sum, the smooth shaft does not provide the desirable rigidity, while the threaded shaft may be vulnerable under high shear stresses present near the facet joint space.

U.S. Pat. No. 6,648,893 (Dudasik) discloses a two-piece facet joint fixation system having a sleeve and an insertion piece. FIG. 3A of Dudasik discloses fenestrations 715 on a proximal portion of the shaft. As Dudasik recites design criteria based upon ASTM F 543-98, Dudasik appears to imply that its system is made of metal. Dudasik does not appear to discloses a resorbable screw.

The general art also discloses several bioabsorbable pins used to fix bony fractures. For example, U.S. Pat. No. 5,180,388 (DiCarlo) discloses a method of using bioabsorbable pins for securing fractured bone fragments in place with tools for measuring hole depth and inserting pins. U.S. Pat. No. 4,898,186 (“Ikada et al.”) discloses an osteosynthetic pin made of poly-L-lactic acid with minimum molecular weight (e.g., ˜70,000 daltons) and axial drawing of 2 to 10 times at elevated temperature (70-120° C.). Another embodiment includes admixing hydroxyapatite into the pin. U.S. Pat. No. 4,858,603 (“Clemow”) discloses a tapered bone pin with a cutting device attached to the small end of the pin for accomplishing drilling and pin insertion in one step. The pin can be bioabsorbable, and is preferably polydioxanone.

In each of DiCarlo, Clemow and Ikada, no mention is made of using the pins in non-fracture situations, such as for preventing motion of the spinal facet joints to augment spinal arthrodesis. Moreover, the essentially smooth nature of the pins disclosed in DiCarlo, Clemow and Ikada provides a less-than-desirable level of rigidity to the fixation.

Claes et al., Biomaterials 17(16):1621-1626 (1996), discloses a resorbable pin adapted for fracture fixation, wherein the shaft of the pin has three interference ridges located in about the center of the shaft. As with DiCarlo, Clemow and Ikada, Claes does not disclose a pin used in facet joint fixation, bur rather for fixation of ankle fractures. In fact, if the Claes pin were used in a facet joint fixation, the central location of Claes' interference ridges could place them directly within the facet joint space during fixation, thereby subjecting those ridges to high shear stresses.

### SUMMARY OF THE INVENTION

It is an object of the present invention to provide a facet joint fixation system that provides initial rigidity for stabilizing the facet joint, is sufficiently strong to withstand facet joint shear stresses, and (after supporting a successful fusion) can be resorbed by the body.

These goals are accomplished by providing a resorbable fixation pin wherein a central portion of the pin is smooth and a proximal portion of the pin is adapted for bony fixation.

The smooth nature of the central portion of the pin will provide strength across of the facet joint, thereby allowing the pin to better withstand the high shear stresses of that joint.

The adaptation of the proximal portion for bony fixation will provide the pin with an enhanced gripping ability, thereby increasing the initial rigidity provided to the functional spinal unit which is required to achieve spinal arthrodesis.

The selection of a resorbable polymer as a material of construction will allow the pin to desirably lose strength with time (thereby promoting gradual load sharing with the spinal elements), and to be ultimately absorbed by the body.

Accordingly, in one aspect of the present invention, there is provided a spinal facet joint fixation device having a bone fixation system:
a) a proximal head,
b) a distal tip, and
c) an elongated intermediate shaft portion having an outer surface comprising:
   i) proximal portion having fastening surface, and
   ii) a smooth central portion,
wherein the shaft portion comprises a resorbable material.

Also in accordance with the present invention, there is provided a spinal facet joint fixation device having a spinal facet fixation system:
a) a proximal head,
b) a distal tip, and
c) a substantially cylindrical intermediate shaft portion having an outer surface comprising:
   i) proximal portion having fastening surface, and
   ii) a smooth central portion,
wherein the cylindrical portion comprises a resorbable material.

### DESCRIPTION OF THE FIGURES

FIG. 1 discloses a side view of a first embodiment of a fixation device of the present invention.

FIG. 2 discloses a posterior view of a functional spinal unit having the fixation device of FIG. 1 inserted therein.

FIG. 3 discloses a side view of a second embodiment of a fixation device of the present invention, comprising a first pin, an insertion piece and an attachment cable.

FIG. 4 discloses a posterior view of a functional spinal unit having the fixation device of FIG. 3 inserted therein.

FIG. 5 discloses a top view of a functional spinal unit and a third embodiment of a fixation device of the present invention, comprising a thread on the distal portion of the shaft.

FIG. 6 discloses a top view of a functional spinal unit having the fixation device of FIG. 5 inserted therein.

### DETAILED DESCRIPTION OF THE INVENTION

For the purposes of the present invention, the proximal portion of the shaft represents the proximal third of the shaft, the central portion of the shaft represents the central third of the shaft, and distal portion of the shaft represents the distal third of the shaft. A “fastening surface” is used interchangeably with “a surface adapted for bony fixation”. An “FSU” is a functional spinal unit, including upper and lower vertebrae and an intermediate disc space.

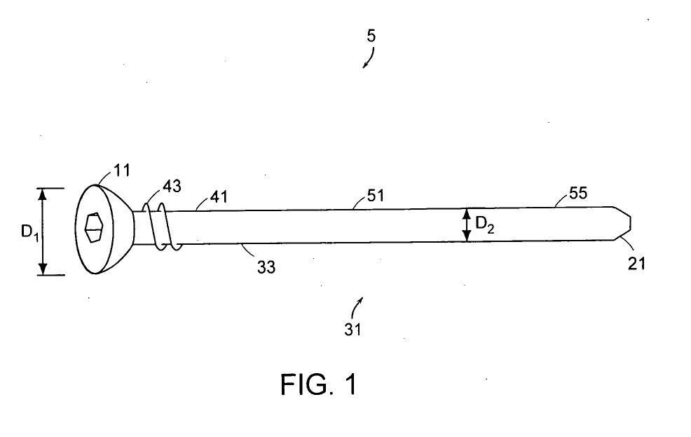

Referring now to FIG. 1, there is provided a bone fixation system 1 comprising a pin 5 comprising:
a) a proximal head 11 having a first maximum diameter D₁,
b) a distal tip 21, and
c) a substantially cylindrical intermediate shaft portion 31 having an outer surface 33 defining a second maximum diameter D₂, the shaft portion comprising:
   i) a proximal portion 41 having fastening surface 43 extending no more than 25% of the shaft length, and
   ii) a smooth central portion 51, and
   iii) a smooth distal portion 55,
      wherein the shaft portion comprises a resorbable material, and
      wherein the first maximum diameter D₁ is greater than the second maximum diameter D₂.

The bone fixation system shown in FIG. 1 has many advantages over the prior art fixation devices.

For example, the absence of threading on the central portion of the shaft increases the strength of the system in this region, thereby providing for a stronger system than the completely threaded PLLA screw of Daguchi in the precise region believed to experience the highest shear stresses. Moreover, the presence of both an enlarged head and threading on the proximal portion of the shaft increases the stiffness of the construct over the completely smooth PLLA pin of Daguchi, thereby enhancing the rigidity of the system in a manner to promote spinal fusion.

In respect of Claes, the absence of threading in the central portion of the shaft reduces the shear stresses, thereby providing for a stronger system than the completely threaded PLLA screw of Claes.

In respect of Dudasik, the resorbable nature of the device of the present invention allows it to gradually load share during fusion and then eliminate itself after fusion is achieved, thereby providing for a more fusion-friendly system than the metallic facet screw of Dudasik. Moreover, the unitary nature of the device of FIG. 1 makes its manufacture and use more simple than the manufacture and use of the Dudasik device.

Accordingly, in one aspect of the present invention, there is provided a spinal facet joint fixation device having a unitary spinal facet fixation system:
a) a proximal head,
b) a distal tip, and
c) a substantially cylindrical intermediate shaft portion having an outer surface comprising:
   i) a proximal portion having fastening surface, and
   ii) a smooth central portion.

The head may have any shape commonly found in fixation devices, including tapered, conical, cylindrical and square. In some embodiment, the head may also have a maximum diameter equal to that of the shaft.

Preferably, however, the head has a maximum diameter D₁ that is larger than the maximum diameter D₂ of the shaft. In these preferred embodiments, the enlarged head provides for better fixation of the device, thereby enhancing the initial rigidity of the FSU.

In some embodiments, the enlarged head has a taper that narrows as it extends distally towards the shaft. This feature allows the head to seat gradually upon the bore formed in the patient's spinous process, thereby minimizing the chances of fracturing this fragile region of bone. More preferably, the taper is a frustocone.

The distal tip of the pin may have any shape commonly found in the tip of fixation devices, including tapered, conical, flat or threaded. It may also have the diameter equal to that of the shaft. Typically, however, the distal tip is narrowed, thereby providing an easier entry into the bore formed in the patient's spinous process.

Preferably, the shaft offers means of providing torsion and shear resistance in a functional spinal unit. In preferred embodiments, the proximal portion of the shaft is adapted for bony fixation, thereby enhancing the rigidity of the FSU. In preferred embodiments, the central portion of the shaft is smooth, thereby enhancing the strength of the pin in a high stress region.

In preferred embodiments, the distal portion of the shaft is smooth. Since tapping of the entire length of the drill hole is contemplated, the smooth distal surface of the shaft minimizes shear stresses upon insertion.

Accordingly, in one aspect of the present invention, there is provided a spinal facet joint fixation device having bone fixation system:
a) a proximal head,
b) a distal tip, and
c) a substantially cylindrical intermediate shaft portion having an outer surface comprising:
   i) a proximal portion having a fastening surface, and
   ii) a smooth central portion, and
   iii) a smooth distal portion.

In preferred embodiments, the length of the shaft is between 1 and 10 cm. In this range, fixation device has an overall length conventionally suited for trans-laminar facet fixation, wherein the proximal end of the device seats upon a first side of the transverse process of a first vertebrae, and the distal end of the device is adjacent the outer surface of the second vertebra (as shown in FIG. 1). In preferred embodiments, the maximum diameter of the shaft is between 2 and 4 mm. In this range, the shaft is strong enough to withstand shear stresses, but small enough to avoid fracture of the spinous process.

In preferred embodiments, the outer surface of the shaft has a fastening surface. Preferably, the fastening surface is located only in the proximal portion of the shaft. When the fastening surface is so located, it provides sufficient rigidity to the FSU while mitigating shear stress issues in the central and distal shaft portions. In some embodiments, the fastening surface is located only in the proximal half of the proximal portion of the shaft. In some embodiments, the fastening surface is located only in the proximal-most quarter of the proximal portion of the shaft.

Although the material of construction of the fixation device can be selected from any suitable biomaterial (including metals, ceramics and polymers), in preferred embodiments, the material of construction is a bioabsorbable polymer. Since the device is intended to act as a temporary stabilization system for spine fusion procedures, the bioabsorbable nature of the polymer will allow a gradual load sharing and transfer during fusion and complete elimination once fusion has been achieved and the device is no longer needed. The bioresorbable nature of this device will also reduce foreign body reaction and facilitate imaging of the spine without artifacts.

In preferred embodiments, the device of the present invention comprises a material of construction that is radiolucent, MRI-compatible and bioabsorbable. Compared to metallic facet and translaminar facet screws, this material does not cause imaging artifacts, reduces the likelihood of long-term foreign body reaction and device-related osteopenia, and does not require removal to allow for growth, particularly in younger patients.

Preferred bioresorbable materials which can be used to make components of the present invention include bioresorbable polymers or copolymers, preferably selected from the group consisting of hydroxy acids, (particularly lactic acids and glycolic acids; caprolactone; hydroxybutyrate; dioxanone; orthoesters; orthocarbonates; and aminocarbonates. Preferred bioresorbable materials also include natural materials such as chitosan, collagen, cellulose, fibrin, hyaluronic acid; fibronectin, and mixtures thereof. However, synthetic bioresorbable materials are preferred because they can be manufactured under process specifications which insure repeatable properties.

A variety of bioabsorbable polymers can be used to make the device of the present invention. Examples of suitable biocompatible, bioabsorbable polymers include but are not limited to polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as collagen, elastin, bioabsorbable starches, etc.) and blends thereof. For the purpose of this invention aliphatic polyesters include, but are not limited to, homopolymers and copolymers of lactide (which includes lactic acid, D-,L- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, χ-butyrolactone, ε-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one, 2,5-diketomorpholine, pivalolactone, χ,χ-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof. Poly(iminocarbonates), for the purpose of this invention, are understood to include those polymers as described by Kemnitzer and Kohn, in the Handbook of Biodegradable Polymers, edited by Domb, et. al., Hardwood Academic Press, pp. 251-272 (1997). Copoly(ether-esters), for the purpose of this invention, are understood to include those copolyester-ethers as described in the Journal of Biomaterials Research, Vol. 22, pages 993-1009, 1988 by Cohn and Younes, and in Polymer Preprints (ACS Division of Polymer Chemistry), Vol. 30(1), page 498, 1989 by Cohn (e.g. PEO/PLA). Polyalkylene oxalates, for the purpose of this invention, include those described in U.S. Pat. Nos. 4,208,511; 4,141,087; 4,130,639; 4,140,678; 4,105,034; and 4,205,399. Polyphosphazenes, co-, ter- and higher order mixed monomer-based polymers made from L-lactide, D,L-lactide, lactic acid, glycolide, glycolic acid, para-dioxanone, trimethylene carbonate and E-caprolactone such as are described by Allcock in The Encyclopedia of Polymer Science, Vol. 13, pages 31-41, Wiley Intersciences, John Wiley & Sons, 1988 and by Vandorpe, et al in the Handbook of Biodegradable Polymers, edited by Domb, et al, Hardwood Academic Press, pp. 161-182 (1997). Polyanhydrides include those derived from diacids of the form HOOC—C₆H₄—O—(CH₂)ₘ—O—C₆H₄—COOH, where m is an integer in the range of from 2 to 8, and copolymers thereof with aliphatic alpha-omega diacids of up to 12 carbons. Polyoxaesters, polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213; 5,700,583; and 5,859,150. Polyorthoesters such as those described by Heller in Handbook of Biodegradable Polymers, edited by Domb, et al, Hardwood Academic Press, pp. 99-118 (1997).

Preferably, the bioresorbable material is selected from the group consisting of poly(lactic acid) (“PLA”) and poly(glycolic acid)(“PGA”), and copolymers thereof. These materials are preferred because they possess suitable strength and biocompatibility, display desirable resorption profiles, and have a long history of safe in vivo use. In general, PLA is a desirable because it typically has a resorption time exceeding 12 months, whereas PGA resorbs fairly quickly (having a resorption time of less than 12 months).

Preferably, the bioresorbable component retains at least 50% of its tensile strength 6 months after implantation, but loses at least 50% of its tensile strength within 24 months of implantation. When this window of bioresorption is achieved, the component has the strength necessary to carry out its intended purpose during the time when bony fusion is occurring, but also bioresorbs after such fusion normally takes place. Also preferably, the bioresorbable polymer retains at least 50% of its mass 6 months after implantation, but loses at least 90% of its mass within 4 years of implantation. This may be accomplished by use of a PLA/PGA copolymer comprising at least 85% PLA.

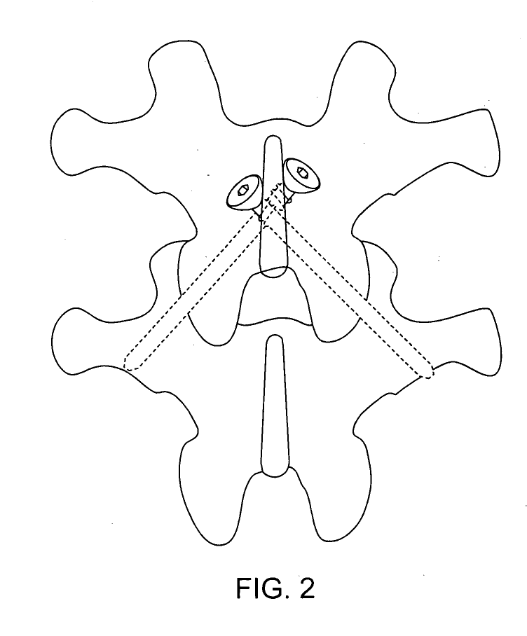

Referring now to FIG. 2, the fixation system of the present invention can be used to translaminarly fix a facet joint. In this method, the tip of a first fixation pin of the present invention is first inserted at the surface of the upper spinous process contralateral from the facet joint to be instrumented. The tip of the pin is then pushed forward to pass within the lamina, cross the facet joint and end in the pedicle. A second pin is similarly inserted through the remaining facet joint of the FSU to provide bilateral fixation. The resulting instrumentation is shown in FIG. 2.

In addition, the fixation system of the present invention can be used to fix a facet joint in a more simple fashion. In this case (not shown), the respective pins are inserted bilaterally through the dorsal side of the facet, across the facet joint and into the pedicle.

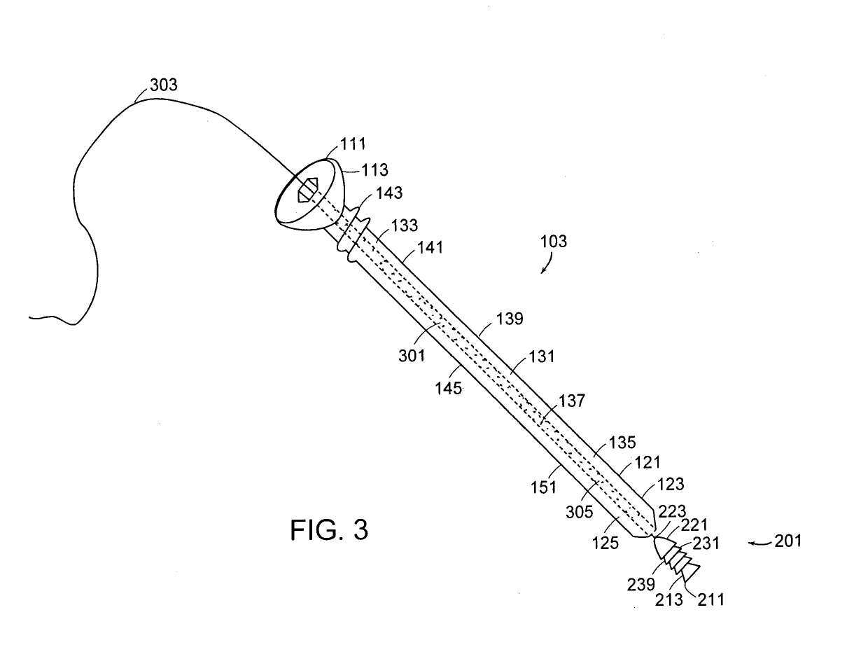

Now referring to FIG. 3, there is provided a second bone fixation system 101 comprising:
a first pin 103 comprising:
   a) a proximal head 111 having a distally narrowing taper 113,
   b) a distal end portion 121 comprising opposing first 123 and second 125 deformable legs, and
   c) a substantially cylindrical intermediate shaft portion 131 having a proximal end 133, a distal end 135, an inner bore 137 and an outer surface 139, the outer surface comprising:
      i) a proximal portion 141 having fastening surface 143,
      ii) a smooth central portion 145, and
      iii) a smooth distal portion 151, and
an insertion pin 201 adapted to fit within the bore and comprising:
   a) a distal head 211 having a distally narrowing taper 213,
   b) a proximal end portion 221 comprising a distally narrowing tip 223, and
   c) a substantially cylindrical intermediate shaft portion 231 having a threaded outer surface 239, and
an absorbable cable 301 having a proximal end portion 303 and a distal end portion 305, wherein the distal end portion is connected to the proximal end of the insertion pin.

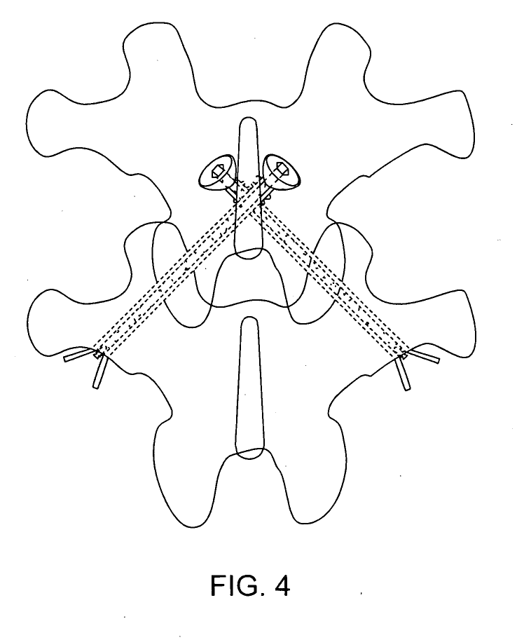

Still referring to FIG. 3, prior to insertion, the proximal end of the cable is first pulled slightly taught so that the proximal end of the insertion pin is lightly seated in the distal end of the bore of the first pin. Now referring to FIG. 4, this assembly is then inserted through the spinous process and across the facet joint as described above, and then further until the tip exits the facet. Third, the proximal end of the cable is pulled with a force sufficient to draw the insertion pin proximally into the bore of the first pin, thereby seating the insertion pin and causing a splaying of the deformable legs. The splaying of the deformable legs causes a proximally directed force upon the lower vertebral body, producing an upward movement of the vertebral body that effectively closes the facet joint, thereby providing secure fixation of the device. By closing the facet joint, the system of the present invention provides for desirable load sharing with the closed joint (rather than the pin taking most of the load).

Accordingly, in one aspect of the present invention, there is provided a spinal facet joint fixation device having a spinal facet fixation system comprising:
a) a proximal head,
b) a distal tip, and
c) a substantially cylindrical intermediate tubular shaft portion defining a longitudinal bore,
wherein the cylindrical portion comprises a resorbable material.

In some embodiments, fixation of the pin can be accomplished by providing a fastening surface (such as a threaded portion as shown) on the distal end of the shaft while providing smooth surfaces on both the central and proximal portions of the shaft.

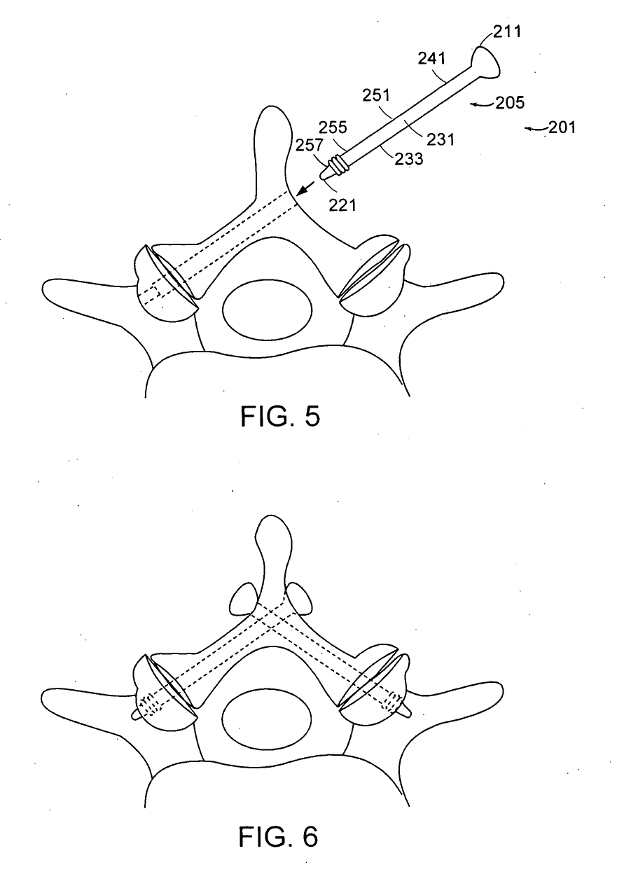

Referring now to FIG. 5, there is provided a bone fixation system 201 comprising a pin 205 comprising:
a) a proximal head 211 having a distally narrowing region,
b) a distal tip 221,
c) an elongated (and, preferably substantially cylindrical) intermediate shaft portion 231 having an outer surface 233, the shaft portion comprising:
   i) a smooth proximal portion 241,
   ii) a smooth central portion 251, and
   iii) a distal portion 255 having a fastening surface 257.

In some embodiments, the shaft comprises a resorbable material. Preferably, the entire pin consists essentially of at least one resorbable material.

Preferably, a dual-diameter bore is drilled into the target bone area prior to insertion of the fixation system of FIG. 5. Now referring to FIG. 6, there is provided a bore having a small distal diameter and a larger proximal diameter. The small distal diameter of the bore is pre-determined to correspond with the small diameter of the threaded distal portion of the shaft of FIG. 6, while the larger proximal diameter of the bore is pre-determined to correspond with the larger diameter of the smooth proximal portion of the shaft of FIG. 6.

The method of implantation of the device is preferably percutaneous and preferably simple and rapid.

Accordingly, in one aspect of the present invention, there is provided a spinal facet joint fixation device having a method of fixing a facet joint, comprising the steps of:
a) providing a bone fixation system:
   a proximal head,
   a distal tip, and
   an elongated intermediate shaft portion having an outer surface comprising:
      i) proximal portion having a fastening surface, and
      ii) a smooth central portion,
         wherein the shaft portion comprises a resorbable material, and
b) inserting the proximal head of the system through the facet joint.

The proposed invention would minimize the damage caused to these supporting elements, reduce surgery time, reduce time for rehabilitation, and therefore reduce greatly the cost of the treatment.

1. A bone fixation system:
a) a proximal head,
b) a distal tip, and
c) an elongated intermediate shaft portion having an outer surface comprising:
i) a proximal portion having fastening surface, and
ii) a smooth central portion,
wherein the shaft portion comprises a resorbable material.

2. The system of claim 1 wherein the fastening surface comprises a thread.

3. The system of claim 1 wherein the fastening surface is located only in the proximal half of proximal portion of the shaft.

4. The system of claim 1 wherein the outer surface of the shaft further comprises:
iii) a smooth distal portion.

5. The system of claim 1 wherein the proximal head is distally tapered.

6. The system of claim 1 wherein the system is unitary.

7. The system of claim 1 wherein the shaft is cannulated, defining a longitudinal bore therein.

8. The system of claim 7 wherein the distal end of the shaft forms first and second deformable legs.

9. The system of claim 8 further comprising:
an insertion pin disposed within the bore.

10. The system of claim 9 further comprising an absorbable cable having a distal end connected to the proximal end of the insertion piece.

11. A spinal facet fixation system:
a) a proximal head,
b) a distal tip, and
c) a substantially cylindrical intermediate shaft portion having an outer surface comprising:
i) a proximal portion having fastening surface, and
ii) a smooth central portion, wherein the cylindrical portion comprises a resorbable material.

12. The system of claim 11 wherein the fastening surface comprises a thread.

13. The system of claim 11 wherein the fastening surface is located only in the proximal half of proximal portion of the shaft.

14. The system of claim 11 wherein the outer surface of the shaft further comprises:
iii) a smooth distal portion.

15. The system of claim 11 wherein the proximal head is distally tapered.

16. The system of claim 11 wherein the system is unitary.

17. The system of claim 11 wherein the shaft is cannulated, defining a longitudinal bore therein.

18. The system of claim 17 wherein the distal end of the shaft forms first and second deformable legs.

19. The system of claim 18 further comprising:
an insertion pin disposed within the bore.

20. The system of claim 19 further comprising an absorbable cable having a distal end connected to the proximal end of the insertion piece.

21. A unitary spinal facet fixation system:
a) a proximal head,
b) a distal tip, and
c) a substantially cylindrical intermediate shaft portion having an outer surface comprising:
i) a proximal portion having fastening surface, and
ii) a smooth central portion.

22. The system of claim 21 wherein the fastening surface comprises a thread.

23. The system of claim 21 wherein the fastening surface is located only in the proximal half of proximal portion of the shaft.

24. The system of claim 21 wherein the outer surface of the shaft further comprises:
iii) a smooth distal portion.

25. The system of claim 21 wherein the proximal head is distally tapered.

26. The system of claim 21 wherein the shaft portion comprises a resorbable material.

27. The system of claim 21 wherein the fastening surface is located only in the proximal half of proximal portion of the shaft.

28. The system of claim 21 wherein the shaft portion comprises a resorbable material adapted to retain at least 50% of its tensile strength 6 months after implantation.

29. The system of claim 21, wherein the bioresorbable material loses at least 50% of its tensile strength within 12 months of implantation.

30. The system of claim 21 wherein the bioresorbable material comprises PLA.

31. A spinal facet fixation system comprising:
a) a proximal head,
b) a distal tip, and
c) a substantially cylindrical intermediate tubular shaft portion defining a longitudinal bore,
wherein the cylindrical portion comprises a resorbable material.

32. The system of claim 31 wherein the shaft portion comprises an outer surface having a fastening surface.

33. The system of claim 32 wherein the fastening surface is located only in a proximal portion of the shaft portion.

34. The system of claim 33 wherein the fastening surface comprises a thread.

35. The system of claim 32 wherein the fastening surface is located only in a proximal half of the proximal portion of the shaft.

36. The system of claim 32 wherein the outer surface of the shaft further comprises:
iii) a smooth distal portion.

37. The system of claim 32 wherein the proximal head is distally tapered.

38. The system of claim 31 wherein the system is unitary.

39. The system of claim 31 wherein a distal end of the shaft forms first and second deformable legs.

40. The system of claim 39 further comprising:
an insertion pin disposed within the bore.

41. A method of fixing a facet joint, comprising the steps of:
a) providing a bone fixation system:
a proximal head,
a distal tip, and
an elongated intermediate shaft portion having an outer surface comprising:
i) proximal portion having a fastening surface, and
ii) a smooth central portion,
wherein the shaft portion comprises a resorbable material, and
b) inserting the proximal head of the system through the facet joint.

42. The system of claim 41 wherein the fastening surface comprises a thread.

43. The system of claim 41 wherein the fastening surface is located only in the proximal half of proximal portion of the shaft.

44. The system of claim 41 wherein the outer surface of the shaft further comprises:
iii) a smooth distal portion.

45. The system of claim 41 wherein the proximal head is distally tapered.

46. The system of claim 41 wherein the system is unitary.

47. The system of claim 41 wherein the shaft is cannulated, defining a longitudinal bore therein.

48. The system of claim 47 wherein the distal end of the shaft forms first and second deformable legs.

49. The system of claim 48 further comprising:
an insertion pin disposed within the bore.

50. The system of claim 49 further comprising an absorbable cable having a distal end connected to the proximal end of the insertion piece.

51. A bone fixation system:
a) a proximal head,
b) a distal tip, and
c) a substantially cylindrical intermediate shaft portion having an outer surface comprising:
i) a proximal portion having a fastening surface, and
ii) a smooth central portion, and
iii) a smooth distal portion.

52. The system of claim 51 wherein the fastening surface comprises a thread.

53. The system of claim 51 wherein the fastening surface is located only in the proximal half of proximal portion of the shaft.

54. The system of claim 53 wherein the fastening surface comprises a thread.

55. The system of claim 51 wherein the proximal head is distally tapered.

56. The system of claim 51 wherein the system is unitary.

57. The system of claim 51 wherein the shaft is cannulated, defining a longitudinal bore therein.

58. The system of claim 57 wherein the distal end of the shaft forms first and second deformable legs.

59. The system of claim 58 further comprising:
an insertion pin disposed within the bore.

60. The system of claim 59 further comprising:
an absorbable cable having a distal end connected to the proximal end of the insertion piece.

61. A facet joint fixation system comprising a pin comprising:
a) a proximal head,
b) a distal tip,
c) an elongated intermediate shaft portion having an outer surface, the shaft portion comprising:
i) a smooth portion,
ii) a smooth central portion, and
iii) a distal portion having a fastening surface.
